(19) United States
(12) Patent Application Publication
(10) Pub. No.: US 2001/0046477 A1
(43) Pub. Date: Nov. 29, 2001

(54) Bleaching/cleaning method and apparatus for tooth whitening

(76) Inventor: Herbert Wolfe, Pomona, NY (US)

Correspondence Address: STEINBERG & RASKIN, P.C., 1140 AVENUE OF THE AMERICAS, 15th FLOOR, NEW YORK, NY, 10036-5803, US

(21) Appl. No.: 09/765,798

(22) Filed: Jan. 19, 2001

Related U.S. Application Data
(60) Provisional application No. 60/177,538, filed on Jan. 21, 2000.

(51) Int. Cl. A61K 7/18; A61K 7/20

(52) U.S. Cl. 424/53

(57) ABSTRACT

The invention relates to a method for bleaching and cleaning teeth for whitening purposes that involves the substantially simultaneous mixing of peroxide and an abrasive cleaning agent and application of the mixture to a patient's teeth, and burnishing the mixture onto the enamel of the teeth.

### REFERENCE TO PRIOR APPLICATIONS

This application is a utility application based on and claims priority from U.S. Provisional Patent Application Serial No. 60/177,538 filed on Jan. 21, 2000.

### BACKGROUND OF THE INVENTION

There are many prior art tooth bleaching/whitening systems that utilize various peroxides to whiten teeth. Peroxide bleaches through an oxidation-reduction reaction. Peroxide bleaches teeth with which it comes into contact with, the compounds on the teeth serving as the oxidized reactant and the peroxide serving as the oxidizing agent in the reaction. Peroxide may be applied to teeth using various methods and in various concentrations and mixed with various other ingredients. This process serves to whiten the teeth. Having whiter teeth is considered by many to be an aesthetically desirable trait.

Using a simple tooth whitening method, peroxide, usually carbamide (urea peroxide), is placed upon a patient's teeth, the carbamide rapidly releases oxygen in an oxidation-reduction reaction and bleaches the enamel of the teeth. A popular use of this method is to place a carbamide-bearing compound in a dental tray and apply the tray about the patient's teeth for a length of time ranging from seconds to minutes or even overnight and, if necessary, repeat the process either immediately thereafter or at some later time.

This method has several drawbacks. First, carbamide is an irritant. When used in a dental tray, as described above, the carbamide comes into prolonged contact with the patient's gums, causing discomfort ranging from mild irritation to severe pain, depending upon the individual patient and the concentration of the carbamide. There is also speculation within the dental profession that prolonged exposure to carbamide may have detrimental effects upon gum and dental tissues. Second, the mere placement of a dental tray about the patient's teeth does not facilitate the opening of the enamel rods of the teeth. Under normal conditions, the enamel rods of the teeth are closed. If they are open, the enamel rods can uptake carbamide significantly faster and in larger quantity than when they are closed. Opening the enamel rods facilitates a faster, more thorough whitening effect from the carbamide. Tooth bleaching using only carbamide in a dental tray will likely require several applications to achieve a desired level of whitening due to closed rods. This method is even less effective when used by home consumers rather than by dental professionals because carbamide for home use is generally available only in very small concentrations and thus lacks even the limited whitening capabilities of higher concentration, professional use carbamide.

Another method of peroxide tooth whitening uses a dental laser. Carbamide gel is applied directly to the teeth. A beam from a dental laser is then directed onto the gel and the resulting interaction between the two rapidly activates the oxygen in the gel and thus starts a whitening process.

The laser method has the first disadvantage of requiring a laser. Lasers are expensive devices that are not possessed or easily attained by all dental professionals. Lasers are also dangerous instruments which may only be properly used by dental professionals and thus are not available to the home consumer. A second disadvantage of the laser method is that it has multiple, time-consuming steps. First, the gel must be applied to the teeth. Then, the laser beam must be directed onto each tooth. Also, since the lasering of all the teeth takes time, the gel setting on portions of teeth not being lasered will already begin reducing, resulting in uneven whitening among some of the teeth.

Many methods of opening the enamel rods, and thereby assisting in the uptake of carbamide, are known in the prior art. All of these methods suffer from a variety of disadvantages. One method of facilitating the opening the enamel rods during bleaching is acid etching. An acid of sufficient concentration is applied to the teeth and the chemical action of the acid serves to open the enamel rods of the teeth. After the acid application, carbamide is then applied to the teeth. The disadvantages of acid etching are numerous. Acid of concentration sufficient to open enamel rods is somewhat dangerous. In other words, acid etching can only be applied by a dental professional and not a home consumer. Acid etching increases the sensitivity of teeth, making the patient more susceptible to discomfort during and after the process. Also, the application of acid to the teeth can cause severe irritation to the patient's gums and possible damage to the gum and dental tissues. Further, the acid etching, if not closely monitored, can ultimately cause the destruction of the tooth enamel. Acid etching also has multiple, time-consuming steps. Acid etching is tedious process that requires extreme attention to detail to minimize its potentially dangerous side effects.

Another method of facilitating the opening of the enamel rods during bleaching involves the use of a pre-mixed carbamide and dental abrasive agent mixture. Such a mixture is applied to the teeth and is then burnished onto the teeth using either a powered dental cleaning cup or brush, or scrubbed on with a more prosaic toothbrush. The dental abrasive agent in the mixture of prophy paste, serves to abrade the teeth, which does three things, 1) it opens the enamel rods to facilitate their uptake of the carbamide, 2) it removes stains in the tooth enamel through mechanical scrubbing action, and 3) it polishes the tooth enamel through mechanical buffing action. Fluoride can be included in the dental abrasive agent. In such a mixture, fluoride decreases tooth sensitivity, thus making the whitening process more comfortable to the patient. This method has the disadvantage of delivering weakened carbamide to the teeth, resulting is less effective whitening. Most readily and economically available carbamides are highly unstable oxidizing agents which lose much of their oxygen (reduce) when exposed to air and when mixed with other ingredients such as prophy paste for an extended period of time. Without oxygen, carbamides can no longer act as oxidizing agents and therefore cannot bleach. Pre-mixing carbamide with prophy paste causes the carbamide to reduce via its mixture with prophy paste if it is not used within a short time thereafter. Thus, the effectiveness of pre-mixed peroxide as a tooth whitener is limited.

### OBJECTS AND SUMMARY OF THE INVENTION

It is an object of this invention is to provide a safe, effective and rapid tooth whitening system.

It is another object of this invention is to provide a tooth whitening system that minimizes patient irritation and injury.

It is a further object of this invention is to provide an easy to use tooth whitening system.

It is still another object of this invention is to provide a tooth whitening system that effectively whitens, as well as cleans and polishes.

It is yet a further object of this invention to provide even whitening among the various teeth being whitened at a given time.

Briefly, in accordance with the present invention, these and other objects are attained by providing an improvement in the abrading technique, a method for bleaching and cleaning teeth which comprises providing quantities of peroxide and an abrasive cleaning agent, mixing the two together to form a mixture and, substantially immediately after mixing, applying the mixture to the teeth to be bleached. Applying the mixture can include placing the mixture into a holding container, removing the mixture from the holding container with a powered dental cleaning cup and applying the mixture to the teeth to be bleached using the same cup.

An apparatus for practicing this method is a two barrel (container) applicator device, similar in configuration to a caulking gun. The applicator contains peroxide and an abrasive cleaning agent in separate barrels. The barrels are sealed to prevent exposure of the peroxide to both the abrasive cleaning agent and air, both of which serve to reduce the oxygen content of the peroxide and its ability to act as an oxidizing or bleaching agent. The applicator also has a mixing applicator tip, which is connected to an end of each barrel, that mixes the peroxide and abrasive cleaning agent as they are being extruded onto the patient's teeth. Keeping the peroxide and abrasive cleaning agent apart from each other and from exposure to air until ready to use creates an ideal situation.

The peroxide is mixed with the abrasive cleaning agent in the mixing applicator tip, exposed to air only as it is extruded from the mixing applicator tip into a holding container. From the container, the mixture can be applied to the teeth, preferably immediately after extrusion or at most shortly thereafter. This allows the peroxide to retain most of its oxygen, and thereby its bleaching strength, until it is actually placed on the teeth to be bleached. Alternately, the peroxide is completely mixed with the abrasive cleaning agent such that the peroxide and the abrasive cleaning agent are evenly distributed throughout the mixture. This insures that the mixture is consistent, resulting in even cleaning and whitening of the various teeth.

The abrasive cleaning agent cleans and opens the enamel rods while the carbamide is also applied to the rods at the same time that they are being opened. This allows maximum peroxide uptake before the rods begin to close. Upon application to the teeth, the mixture is burnished into the tooth using the powered dental cleaning cup. This process, like the normal application of prophy paste, spreads very little excess mixture so that it cannot come into contact with the gums and cause irritation.

The abrasive cleaning agent, through mechanical abrasion, causes the immediate opening of the enamel rods in the teeth. Simultaneously, the open enamel rods rapidly and voluminously uptake the highly-oxygenated carbamide and fast, effective bleaching results. The burnishing of the mixture's abrasive cleaning agent also results in scrubbing stains from and polishing the enamel of the teeth, further enhancing both the health and appearance of the teeth. The apparatus is operated by a hand-actuated mechanism which incrementally forces dual plungers into the barrels containing the peroxide and the abrasive cleaning agent and incrementally causes their mixing and extrusion of the mixture. The amount of mixture extruded can be easily controlled by the operator (dental professional).

The method and apparatus of the invention may also be practiced by the home consumer. A paste similar to toothpaste but containing an abrasive can be used in place of a professional abrasive cleaning agent, such as prophy paste. A firm bristle toothbrush, electric toothbrush or home dental cleaning/polishing cup can be used in place of a powered dental cleaning cup. The peroxide used may be of lower concentration than professional-grade peroxide and the paste may be less abrasive than prophy paste to satisfy pharmaceutical standards or other safety concerns. This alternate home method, by virtue of its potentially lower concentration of peroxide, its less abrasive paste and its less vigorous brushing mechanism, may be less effective than the preferred embodiment of the method. However, it is still a very effective method of whitening teeth when used as a part of a dental hygiene regimen.

### DESCRIPTION OF THE DRAWINGS

A more complete appreciation of the present invention and many of the attendant advantages thereof will be readily understood by reference to the following detailed description when considered in connection with the accompanying drawings in which:

FIG. 1 is a perspective view of an embodiment of apparatus in accordance with the invention utilized in the preferred embodiment of the method of the invention;

FIG. 2 is a front elevation, exploded view of the components of the apparatus shown in FIG. 1;

FIG. 3 is a perspective view of the apparatus of FIG. 1 illustrating the addition of the container component to the applicator;

FIG. 4 is a perspective view of the apparatus of FIG. 1 in use prior to mixing and application of the mixture; and

FIG. 5 is a perspective view of the apparatus being utilized during simultaneous mixing and application in accord with the preferred embodiment of the method of the invention.

### DETAILED DESCRIPTION OF THE PREFERRED EMBODIMENT

In accordance with the present invention, a method for bleaching, cleaning and teeth whitening comprises the steps of providing a quantity of peroxide, providing a quantity of abrasive cleaning agent, completely mixing a portion of said peroxide with a portion of said abrasive cleaning agent to form a mixture wherein the portion of peroxide and the portion of abrasive cleaning agent are evenly distributed, substantially immediately after the mixture step placing/applying the mixture into a holding container, removing the mixture from the container with a powered dental cleaning cup and using this cup to apply the mixture to at least one tooth. This method provides safe, rapid tooth whitening that also cleans and polishes teeth. The method can be practiced by dental professionals and home consumers while minimizing the potential for patient injury and irritation.

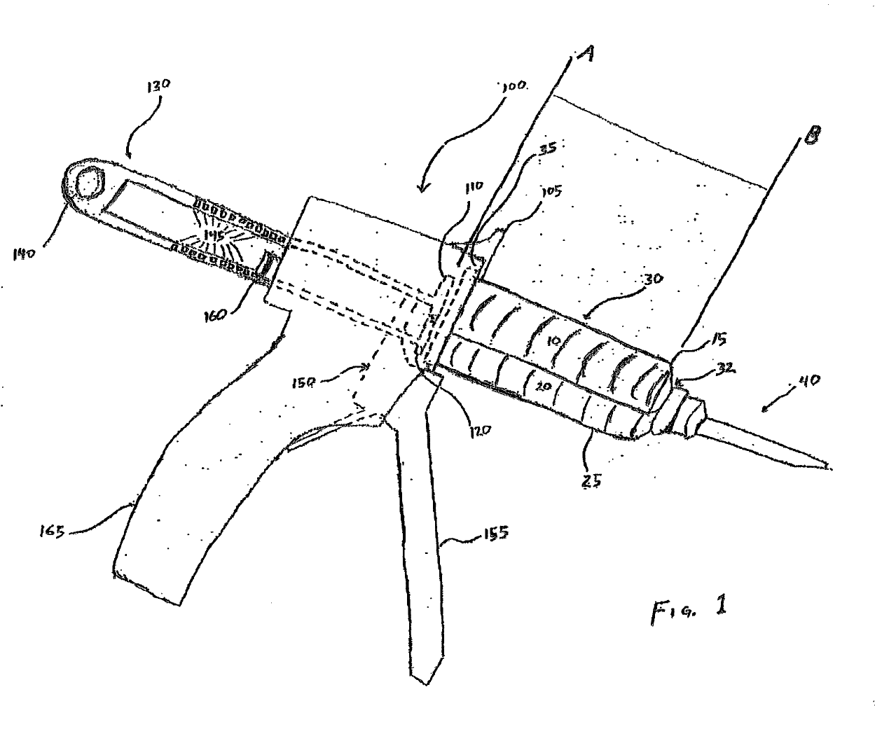

Referring to FIGS. 1-5, a preferred embodiment of an apparatus for practicing the method of the invention comprises an applicator loaded with a container unit which contains the peroxide, i.e., carbamide, and the abrasive cleaning agent, i.e., prophy paste with fluoride, which is applied using a mixing applicator tip. Container unit 30 comprises a first barrel 15 and a second barrel 25 which are joiningly attached to each other on their respective exteriors for ease of handling. Container unit 30 is removably attachable at a plunging end 35 to dual plunger applicator 100 at ends of a pair of plungers 110 and 120. Container unit 30 is secured into applicator 100 by locking mechanism 105. Plungers 110 and 120 are formed as a single plunger unit 130 interconnected by a grip 140 having a plurality of ratchet teeth 145. Applicator 100 also has a ratcheting mechanism 150 with actuation lever 155, release 160 and handle 165. Ratcheting mechanism 150 engages ratchet teeth 145. Container unit 30 is connected at an orifice end 32 to a mixing applicator tip 40.

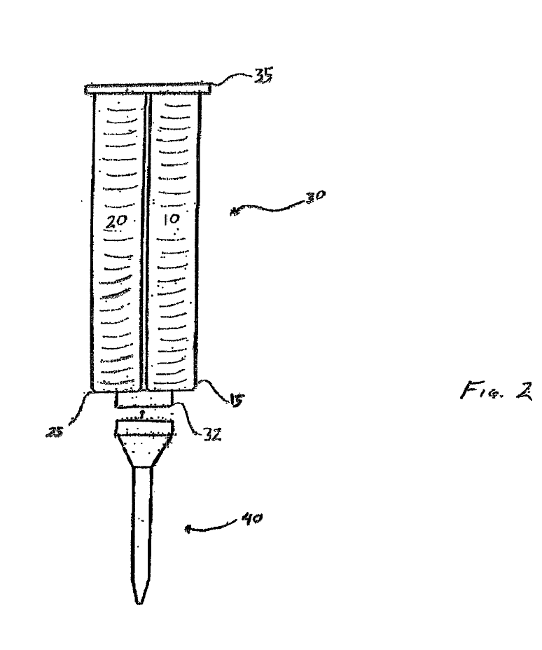

As shown in FIG. 2, the bleaching and cleaning method for tooth whitening comprises placing carbamide 10 into barrel 15 and fluoridated prophy paste 20 into barrel 25. This placement preferably takes place under controlled, factory conditions to avoid spillage, contamination and exposure of the carbamide to air. The placement may alternately take place under sanitary conditions in a dentist's office, wherein the method of the invention is still practiced but without the advantage of avoiding any contact between the carbamide and air. Mixing applicator tip 40 is attached to orifice 32 of container unit 30.

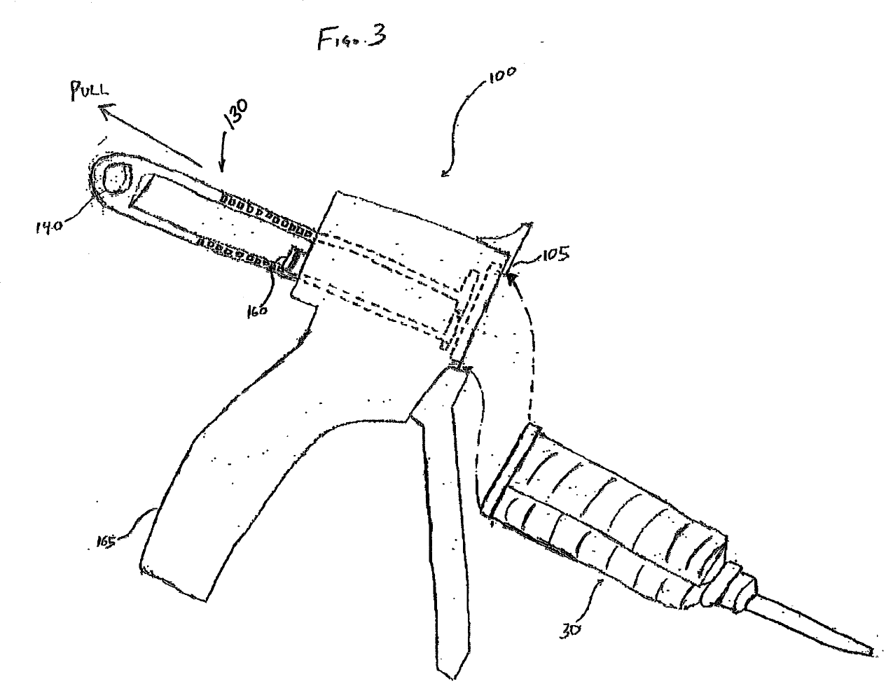

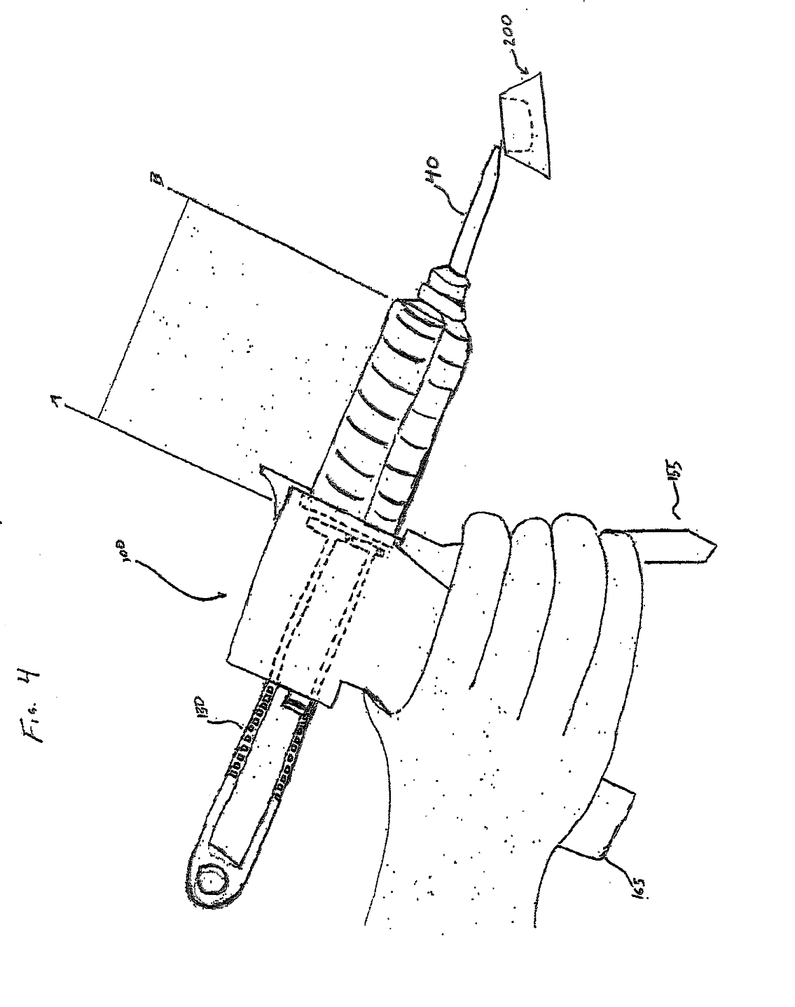

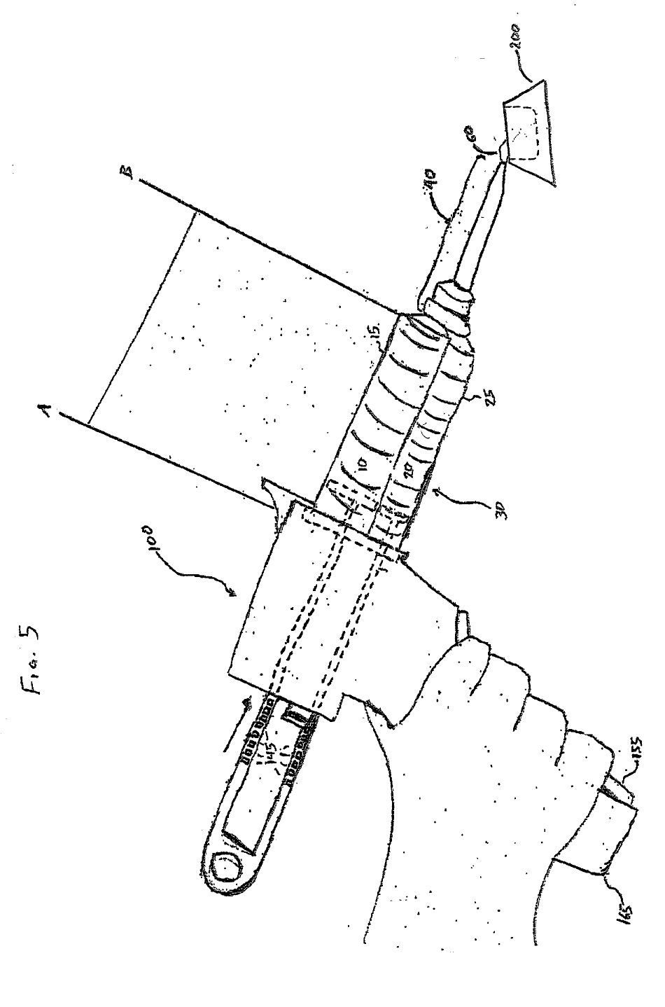

Next, as shown in FIGS. 3 and 4, plunger unit 130 is placed in its retracted position A. This is accomplished by pressing release 160 while pulling grip 140 away from mixing applicator tip 40. The filled container unit 30 is then attached to applicator 100 at plunging end 35 and secured thereto by locking mechanism 105. As shown in FIG. 4, applicator unit 100 is then gripped around handle 165 and actuation lever 155 by a user's hand. The applicator unit 100 is then placed in proximity to a dappen dish 200. As shown in FIG. 5, the user then squeezes the actuation lever 155 and the handle 165 together. This ratchets ratchet teeth 145 and causes plunger unit 130 to move toward mixing applicator tip 40 (extended position B), applying constant, equal and controlled pressure to carbamide 10 and prophy paste 20 in separate barrels 15 and 25 of container unit 30 respectively. The actuation is repeated until the pressure is sufficient to force an amount of carbamide 10 and prophy paste 20 into and through mixing applicator tip 40, wherein they are completely mixed together.

The mixture 60 is extruded from mixing applicator tip 40 into dappen dish 200. A dental cleaning cup is then immediately dipped into dappen dish 200 and mixture 60 is removed therefrom. The mixture is burnished onto the teeth with the dental cleaning cup in the same manner in which prophy paste alone is normally burnished onto teeth for cleaning (not shown).

In alternate embodiments, the dental cleaning cup may be replaced by a number of other mechanical cleaning implements. These include but are not limited to a firm bristle toothbrush, an electric tooth brush and a home dental cleaning/polishing cup. Also, in alternate embodiments, the applicator may be replaced by a number of other application implements. These include but are not limited to a plunger apparatus similar to those used to mix and apply epoxy.

Obviously, numerous modifications and variations of the present invention are possible in light of the above teachings. Accordingly, it is understood that other embodiments of the invention are possible in the light of the above teachings.

I claim:

1. A bleaching and cleaning method for tooth whitening, comprising the steps of:
providing a quantity of peroxide;
providing a quantity of abrasive cleaning agent;
mixing a portion of said peroxide with a portion of said abrasive cleaning agent to form a mixture;
substantially immediately after said mixing step, applying the mixture on at least one tooth.

2. The method of claim 1, wherein said step of applying comprises:
placing the mixture into a holding container;
removing the mixture from within the holding container with a tool; and
applying the mixture on said at least one tooth using the tool.

3. The method of claim 2, wherein said tool is a powered dental cleaning cup.

4. The method of claim 3, further comprising burnishing the mixture on the tooth using said dental cleaning cup.

5. The method of claim 2, wherein said tool is a toothbrush.

6. The method of claim 5, further comprising the step of:
scrubbing the mixture on said at least one tooth using said toothbrush.

7. The method of claim 1, wherein said step of mixing is complete such that the portion of peroxide and the portion of abrasive cleaning agent are distributed evenly throughout the mixture.

8. The method of claim 1, wherein said step of providing a quantity of peroxide encompasses providing said quantity such that it has not been exposed to air prior to said mixing step.

9. The method of claim 1, wherein said holding container is a dappen dish.

10. The method of claim 1, wherein the peroxide is carbamide.

11. The method of claim 1, wherein the abrasive cleaning agent contains fluoride.

12. The method of claim 1, wherein the abrasive cleaning agent is prophy paste.

13. The method of claim 1, wherein the abrasive cleaning agent is paste containing an abrasive.

14. A bleaching and cleaning method for tooth whitening comprising the steps of:
placing peroxide into a first container having at least one orifice;
placing abrasive cleaning agent into a second container having at least one orifice;
extruding a portion of said peroxide and a portion of said abrasive cleaning agent through their respective orifices into a mixing applicator tip;
mixing the peroxide and the abrasive cleaning agent in the mixing applicator tip to form a mixture,
extruding said mixture from the mixing applicator tip; and
substantially immediately after said mixing step, applying the mixture on at least one tooth.

15. The method of claim 14, wherein said step of applying comprises:
placing the mixture into a holding container;
removing the mixture from within the container with a tool; and
applying the mixture on at least one tooth using the tool.

16. The method of claim 15, wherein said tool is a powered dental cleaning cup.

17. The method of claim 16, further comprising burnishing the mixture on the tooth using said dental cleaning cup.

18. The method of claim 15, wherein said tool is a toothbrush.

19. The method of claim 18, further comprising scrubbing the mixture on the tooth using said toothbrush.

20. The method of claim 14, wherein said step of mixing is complete such that the peroxide and the abrasive cleaning agent are distributed evenly throughout the mixture.

21. The method of claim 14, wherein said holding container is a dappen dish.

22. The method of claim 14, further comprising the step of:
burnishing the mixture on the tooth using a dental cleaning cup.

23. The method of claim 14, further comprising the step of
scrubbing the mixture on the tooth using a toothbrush.

24. The method of claim 14, wherein the peroxide is carbamide.

25. The method of claim 14, wherein the abrasive cleaning agent contains fluoride.

26. The method of claim 14, wherein the abrasive cleaning agent is prophy paste.

27. The method of claim 14, wherein the abrasive cleaning agent is paste containing an abrasive.
